(19) United States
(12) Patent Application Publication
(10) Pub. No.: US 2013/0291622 A1
(43) Pub. Date: Nov. 7, 2013

(54) GAS FLUX MEASUREMENT SYSTEM AND METHOD

(71) Applicant: Andreas Heinemeyer, Heslington (GB)

(72) Inventor: Andreas Heinemeyer, Heslington (GB)

(73) Assignee: LI-COR, INC., Lincoln, NE (US)

(21) Appl. No.: 13/820,475

(22) PCT Filed: Sep. 12, 2011
(86) PCT No.: PCT/GB2011/001330

§ 371 Date: Jun. 4, 2013

(30) Foreign Application Priority Data
Sep. 10, 2010 (GB) 1015047.2

(51) Int. Cl. G01N 33/00

(52) U.S. Cl. CPC G01N 33/0004
USPC 73/23.2

(57) ABSTRACT

A gas flux measurement system comprises gas collection means, operable to collect gas for analysis, and gas analysing means, operable to analyse the collected gas. The gas collection means has at least one elongate tube. The, or each, tube has one or more gas collection openings to provide gaseous communication from outside of the tube to the inside of the tube.

### CLAIM TO PRIORITY APPLICATIONS

The present disclosure claims priority to PCT Application No. PCT/GB2011/001330, filed 12 Sep. 2011, which claims priority to United Kingdom Patent Office Application No. GB 1015047.2, filed 10 Sep. 2010.

### BACKGROUND

Unless otherwise indicated herein, the approaches described in this section are not prior art to the claims in this application and are not admitted to be prior art by inclusion in this section.

The present invention relates to a system and method for measuring gas flux and particularly to a system and method for measuring gas flux emitted from a surface.

Gas flux can be defined as the amount of a gas emitted from a surface area over a period of time.

For example, CO2 flux emitted from soil respiration into the atmosphere is estimated to account for almost 10% of the annual global fossil fuel emissions. Globally, it is estimated that soils store three times as much Carbon as the atmosphere. Therefore, due to global warming it is becoming increasingly important to measure and monitor the respiration of CO2 from soil into the atmosphere.

A large global store of soil Carbon is Earth's grasslands, which cover approximately 25% of the terrestrial biosphere. Grasslands are particularly important to monitor as Carbon is additionally released by microbial decomposition which is stimulated by increased temperature. Consequently, future increases in the earth's temperature have the potential to cause a positive feedback and thereby an exponential increase in the release of Carbon into the atmosphere from grasslands. It is therefore desirable for there to be an accurate system of measurement of soil respiration, particularly from grasslands or densely vegetated croplands, without disturbing or removing the vegetation (Kutsch, Bahn, Heinemeyer, 2009).

A known method of measuring gas flux emitted from soil uses a chamber based system. This method involves positioning a chamber to overly a predetermined portion of the soil surface, trapping a volume of air in the chamber and measuring the CO2 in the trapped air over time. However, this method and system is disadvantaged in that it cannot be applied to grasslands or dense croplands because of interference from the vegetation above ground level. Further, disturbing the vegetation or cutting it back in order to position the chamber without interference from the vegetation only results in inaccurate measurements in the grassland and cropland areas as any measurements alter and partly exclude root derived Carbon fluxes because cutting the vegetation cuts the supply of Carbon from the leaves to the roots. Furthermore, the soil environment is significantly changed if the vegetation is disturbed or cut back as, for example, the soil will have less shade and will be more exposed to the wind and therefore will be warmer and drier. Consequently, the measured Carbon flux and its environmental response do not reflect the true natural conditions and therefore provides inaccurate data for flux predictions or model development and validation in grassland and cropland environments

Patent document WO2008/070922 discloses a method and system wherein a flux chamber is buried in the ground where it measures subsurface gas fluxes using appropriate gas sensors disposed within the chamber.

Another known method, referred to as the gradient method, involves burying a number of CO2 sensors in the soil at different depths, sensing and measuring the subsurface gas and calculating diffusion gradients and estimating diffusion coefficients therefrom.

Both these latter methods and systems are disadvantaged in that they cannot be used for accurate measurements in areas of grasslands and croplands as the methods and systems both require the removal of the vegetation and disturbance of the soil. Therefore, the measurements exclude root derived Carbon fluxes because cutting the vegetation cuts the supply of Carbon from the roots to the leaves. Further, the soil environment is significantly changed if the vegetation is disturbed or cut back. For example, the soil will have less shade and therefore will be warmer and drier. Furthermore, these subsurface methods and systems require the chamber/sensors to be buried and therefore the soil itself is disturbed. Consequently, the measured Carbon flux and its environmental response do not reflect the true natural conditions and therefore these methods provide inaccurate data for flux predictions or model development and validation in grassland and cropland environments.

Moreover, the above-mentioned systems are relatively expensive which is currently prohibiting the gas flux analysis required to properly and accurately assess the issue of CO2 respiration into the atmosphere.

### SUMMARY

Although the present invention has been introduced as a gas flux measurement method and system related to the analysis of gas flux in grassland and cropland environments, it is not limited to such environments and is equally applicable to other applications which are described later in this patent application. Moreover, it is not limited to the analyses of CO2 but equally applicable to the analyses of other gases.

It is desirable in this technical field for there to be a method and system which provides accurate gas flux measurements which reflect the true natural state of the environment and a method and system which is capable of providing gas flux measurements from surfaces which to date have not been accurately measurable and/or have been inaccessible using known systems.

It is an object of the present invention to provide a relatively inexpensive system and method of accurate gas flux measurement which overcomes the disadvantages of the above-mentioned known systems and methods.

The invention will now be described in detail with reference to the following drawings, in which:

### BRIEF DESCRIPTION OF THE DRAWINGS

FIG. 1 is a schematic drawing of a gas flux measurement system according to the present invention.

FIG. 2 is a schematic enlarged drawing of the tube as shown in the system of FIG. 1.

FIG. 3 is a schematic drawing of an alternative embodiment of a tube as shown in FIG. 2.

FIG. 4 is a schematic drawing of a plurality of connected tubes of FIG. 2 or 3.

FIG. 5 is a schematic drawing of the tube of FIG. 2 or 3 positioned, in use.

FIG. 6 is a schematic drawing of a preferred embodiment of a gas flux measurement system according to the present invention.

### DETAILED DESCRIPTION

According to a first aspect of the present invention there is provided a gas flux measurement system comprising gas collection means, operable to collect gas for analysis, and gas analysing means, operable to analyse the collected gas, wherein the gas collection means comprises at least one elongate tube, the, or each, tube having one or more gas collection openings to provide gaseous communication from outside of the tube to the inside of the tube.

The system preferably further comprises a semi-permeable membrane disposed to cover the one or more openings such as to allow gas to enter the tube and prevent undesirable fluids and solids entering the tube.

The system preferably further comprises securing means suitable for securing the, or each, tube to a surface.

The tube advantageously comprises a flattened outer portion extending longitudinally along the length of the tube. The one or more gas collection openings are preferably disposed along the length of the flattened outer portion.

The at least one flexible tube is advantageously sufficiently buoyant or is adaptable to provide sufficient buoyancy such that it will at least partially float on, or near to, the surface of a liquid.

The tube is advantageously flexible.

The system advantageously further comprises one or more tube connectors for connecting adjacent tubes in series and/or parallel arrangement.

The gas analysing means advantageously comprises a gas multiplexer.

The system advantageously further comprises a closed loop gas flow tube line and an open loop gas flow line, wherein the open loop gas line is operable to flush the system.

The gas analysing means preferably comprises a plurality of gas valves.

The plurality of gas valves are preferably pinch valves.

One gas valve is preferably associated with the regulation of gas flow in the closed loop line and two gas valves are preferably associated with the regulation of gas flow in the open loop line.

According to a second aspect of the present invention there is provided a gas flux measurement tube comprising one or more gas collection openings to provide gaseous communication from outside of the tube to the inside of the tube.

The gas flux measurement tube may further comprise securing means suitable for securing the tube to a surface.

The gas flux measurement tube advantageously comprises a flattened outer portion extending longitudinally along the length thereof. The one or more gas collection openings are advantageously disposed along the length of the flattened outer portion.

The gas flux measurement tube may be sufficiently buoyant such that it will at least partially float on, or near to, the surface of a liquid.

The gas flux measurement tube advantageously flexible.

The gas flux measurement tube advantageously comprises one or more tube connectors for connecting adjacent tubes in a series or parallel arrangement.

According to a third aspect of the present invention there is provided a method of measuring gas flux comprising the steps of: providing gas collection means comprising at least one elongate tube having one or more gas collection openings; providing gas analysing means; providing gas pumping means; disposing the elongate tube on a surface from which gas flux is to be measured; collecting gas from the said surface and pumping the collected gas through the at least one tube to the gas analysing means; analysing the collected gas and measuring the gas flux.

The method preferably further comprises the step of providing a semi-permeable membrane disposed to cover the one or more openings such as to allow gas to enter the tube and at least substantially prevent undesirable fluids and solids entering the tube.

The method preferably further comprises the steps of providing securing means and securing the, or each, tube to the said surface.

The tube advantageously comprises a flattened outer portion extending longitudinally along the length of the tube and the one or more gas collection openings are disposed along the length of the flattened outer portion, and wherein the step of disposing the tube on the said surface comprises disposing the flattened outer portion against the said surface.

The said surface may have vegetation growing therefrom and accordingly the step of disposing the tube comprises adapting the tube in such a way as to at least substantially mitigate disruption of the said vegetation.

Additionally, or alternatively, the said surface may be a liquid and the step of providing gas collection means advantageously comprises providing a tube having sufficient buoyancy such that it will at least partially float on, or near to, the surface of a liquid and the step of disposing the tube on the surface may comprise floating the tube on the surface.

The method advantageously further comprises the steps of providing one or more tube connectors and connecting adjacent tubes in series and/or parallel arrangement.

The gas analysing means advantageously comprises a gas multiplexer and the method advantageously further comprises connecting a plurality of the at least one tubes to the gas multiplexer and multiplexing gas received therefrom.

The, or each, at least one tube is advantageously arranged to provide a closed loop gas flow tube line and an open loop gas flow tube line, wherein the open loop gas flow tube line is operable to flush the system.

The method advantageously further comprises the step of providing a plurality of gas valves wherein one gas valve is associated with the regulation of gas flow in the closed loop line and two gas valves are associated with the regulation of gas flow in the open loop line.

The gas valves are preferably pinch valves.

The method advantageously further comprises the step of flushing the system prior to the collection of gas.

The method advantageously further comprising the steps of providing timing means, selecting a period of time and flushing the system for the selected period of time.

The invention will now be described in detail with reference to the drawings.

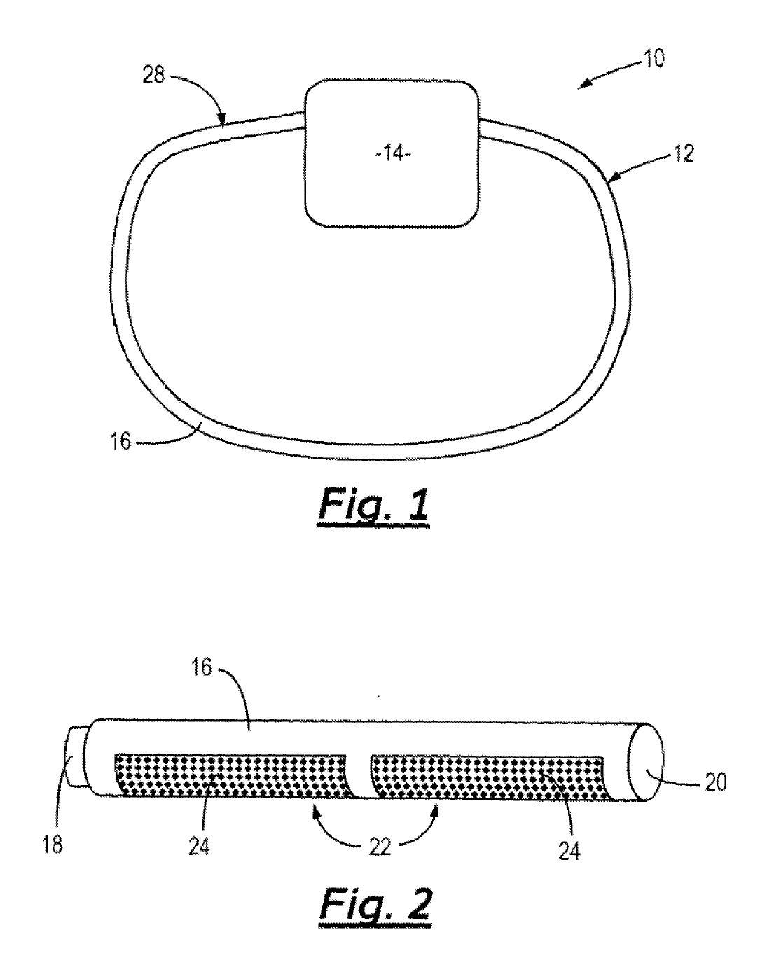

Referring to FIG. 1, a gas flux measurement system 10, according to the present invention, comprises a gas collection means 12 and gas analysing means 14. The gas collection means 12 is formed from one or more elongate tubes 16. The tubes 16 are advantageously flexible and made form, for example, PVC.

Referring also to FIG. 2, in a preferred embodiment of the present invention the system 10 comprises a plurality of the elongate tubes 16 with each tube 16 having tube connecting means such as, for example, gas tight male 18 and female 20 connectors which may be screw threaded or bayonet fitted. The, or each, tube 16 is flexible and has gas collection openings 22 longitudinally disposed in series along its length. The gas collection openings 22 are covered with a semi-permeable membrane 24 such as to allow gas to enter the tube 16 and prevent undesirable fluids and solids entering the tube. A suitable semi-permeable membrane may be, for example, PVDE.

Referring to FIG. 3, an alternative embodiment of the tube 116 has a flattened outer portion 117 having gas collection openings 122, covered by a semi-permeable membrane 124, extending in series longitudinally along the length thereof. In use, the flattened outer portion 117 provides for more stable positioning and better contact of the tube 116 against a surface which can facilitate the collection of gas.

It will be appreciated that either tube 16 or tube 116 may be used in relation to the system and method as described. However, for the benefit of clarity only tube 16 is referred to.

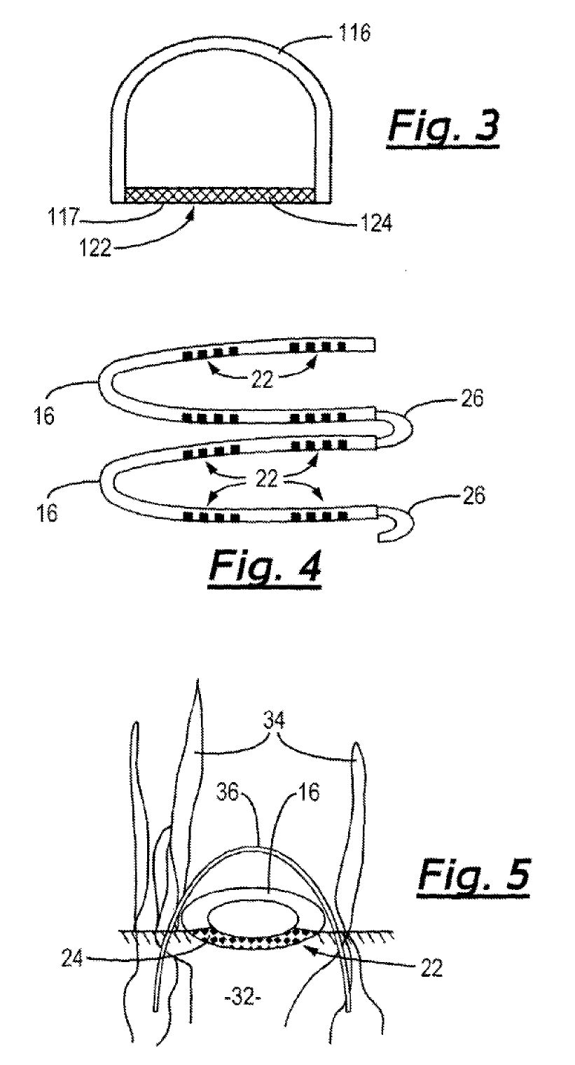

Referring also to FIG. 4, the system 10 further comprises connectors 26 for connecting adjacent tubes 16. The connectors 26 may be suitable for connecting tubes 16 in series, either in a substantially straight line or at a predetermined angle, for example through 180° (as shown in FIG. 4) or tubes 16 in parallel, to provide a network of tubes. The connectors 26 may be, for example, push fit, screw threaded or bayonet fit. In use, the plurality of tubes 16 are connected in series such as to form a closed loop gas flow line 28 which passes through the gas analysing means 14, as shown in FIG. 1.

Referring to FIG. 5, the system further comprises securing means in the form of hooks 36 which are placed over the tubes 16 and into the surface 32, which in this case is the ground. The system of the present invention is particularly advantageous for the collection and analysis of gas flux emitted from ground surfaces which have vegetation, as the tubes 16 can be flexed and interlaced between the vegetation with minimal disturbance. This removes the need to cut back vegetation and provides for more accurate measurements. In use, tubes 16 are positioned against the ground 32 such that they flex and interlace between the vegetation 34 and are secured to the surface of the ground 32 using the hooks 36. The tubes 16 are orientated such that the gas collection openings 22 overlie the surface of the ground 32.

The gas analyser 14 comprises a gas pump and instrumentation for measuring gas flux example, an Infrared Gas Analyser suitable for measuring the flux of a predetermined gas, such as, for example CO2.

In use, traces of CO2 are collected through the gas collection openings 22 of the tubes 16 and pumped around the closed loop gas flow line 28 and are measured by the gas analyser 14. The measured data is then stored.

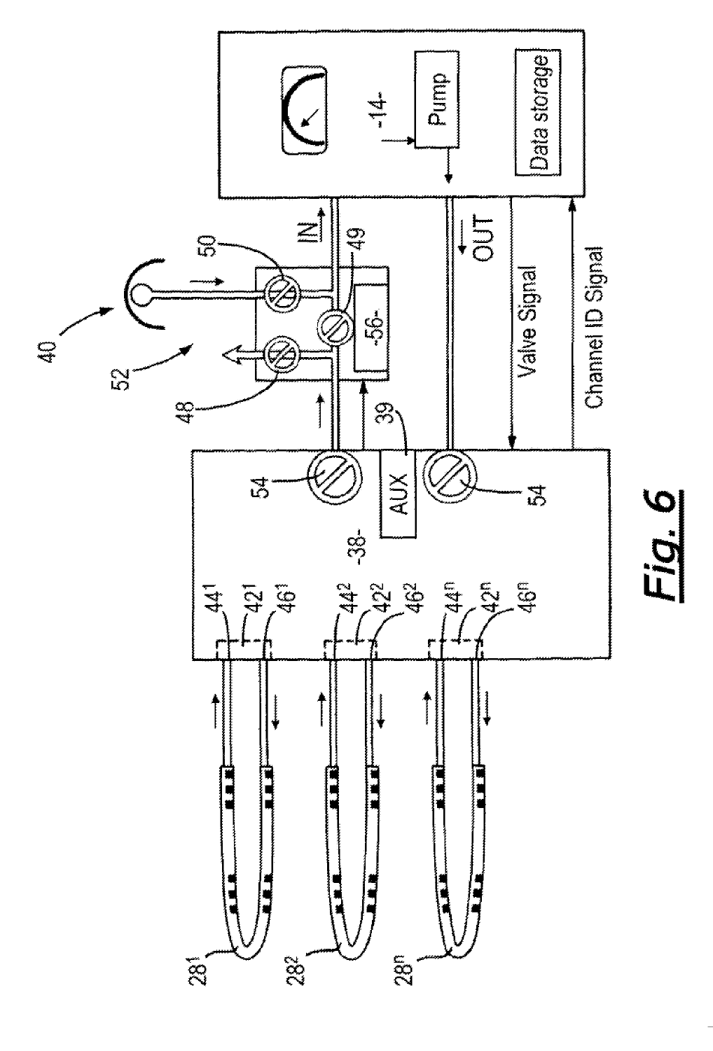

Referring to FIG. 6, a preferred embodiment of the system, according to the present invention, further comprises a gas multiplexer 38 and flushing means 40. The gas multiplexer 38 has a plurality of channels 421 to 42n and an AUX interface 39. The multiplexer typically has sixteen channels but for the benefit of clarity only three channels are shown in FIG. 6. Each channel 42n has an input 44n and an output 46n. Each associated channel input 44n and output 46n is connected to an associated closed loop gas flow line 281 to 28n.

The flushing means 40 comprises valves 48, 49 and 50 which are connected to the multiplexer (through the AUX connector 39) for providing an open loop gas flow line 52 such that each closed loop gas flow line 28n can be independently selected (using the multiplexer) and flushed prior to collecting gas for analyses.

Connecting the flushing means 40 using the AUX connector 39 provides a 12V power source and also a logic input which is high (5V) when a channel is inactive and goes low (0V) when the gas analyser instructs the channel to close. It also allows for four logic inputs providing a binary indication of which channels are currently in use. For example, all four inputs low (0V) represent line 1 (binary 0) and all four inputs high (5V) represent line 16 (binary 15).

A bank of paired closed loop gas flow valves 54 (one pair for each channel 42) regulate the closed loop gas flow, and provide for the selection of individual channels 42 (e.g. 1-16). The valves 48, 49, 50 and 54 are Biochem 100P2 series pinch valves with ¼″ OD, ⅛″ ID tubing. Although it will be appreciated that other valves and tubing may be used within the working of the present invention. The closed loop gas flow valve 49 is “normally” open and the flushing valves 48 and 50 are “normally” closed within the normal mode of working the system according to the present invention.

In a 16-channel multiplexer configuration, the flushing box 40 Timer/Switch controller 56 comprises two rows of eight channel selection switches (not shown). When a switch is in an “ON” position it denotes a closed loop gas flow line 28n is connected to that specific channel 42n on the multiplexer 38.

A further bank of switches (not shown) in Timer/Switch controller 56 provides two further functions. Four valve delay switches set a delay timer, which provides a period of delay before the closed loop valve 49 is closed and flushing valves 48 and 50 are opened and a closed loop gas flow line 28n is flushed. The four switches provide delay time options of +10 seconds, +20 second, +40 seconds and +80 seconds. By switching one or more switches to ON the delay time is selected. Accordingly, a delay of between 10 and 150 seconds can be set. A further two flush period switches set the timer 56 for a flush period. The two switches provide flush periods of +10 seconds and +20 seconds. Accordingly, flush periods of 10, 20 or 30 seconds can be selected. However, it will be appreciated that alternative switches and delay periods could be utilised within the working of the present invention and to suit specific system applications and needs.

In use, a microcontroller inside flushing means 40 continually monitors the close-chamber signal of multiplexer 38 regulating channels 42n, which is taken from the AUX connector 39 on the multiplexer 38. When this signal goes from high (5V) to low (0V), a microcontroller reads the position of the relevant switch from the bank of 16 inside the flushing means box 40 in order to detect whether a closed loop gas flow line 28n is connected (other chamber devices can also be connected to individual gas flow lines), and thus detects whether or not to activate (i.e. change from normal to operational state) the flushing means box valves 48, 49 and 50 after a preset time period in order to purge the gas flow line. It detects which switch to read from reading the state of the four binary logic.

If there is no closed loop gas flow line 28n connected no further action is taken. If a closed loop gas flow line 28n is connected, the pre-flushing delay period is read from the valve delay switches and the timer set. When the delay period has elapsed, the microcontroller reads the flush period from the flush period switches and sets the timer 56 accordingly. At this point, the closed loop gas flow valve 49 is closed and the flushing valves 48 and 50 are opened to provide the flushing function. This continues for the duration of the flushing period at the end of which the flushing valves 48 and 50 return to a closed position and the closed loop gas flow valve 49 returns to an open position.

When for example analysing the CO2 flux of soil, the soil surface CO2 (approximately 1000 ppm) equilibrates with the air in the tube 16. The closed loop gas flow line 28n is then flushed, as described above, with ambient air (i.e. from above or near the surface at approximately 380 ppm) for approximately 10 seconds. The closed loop gas flow line 28n is then closed again and soil CO2 diffuses into the tubing, through the semi-permeable gas collection membrane openings 22, and will reach an equilibrium with soil surface CO2 over approximately 5 minutes. The increase in CO2 over time, during the first 20 to 40 seconds is normally sufficient to calculate the gas flux.

The above-mentioned example relates to the measurement of CO2 flux from soil. However, the system and method, according to the present invention may be applied to the monitoring and measurement of flux of other gases such as, for example, methane and nitrous oxides, and from different surfaces and ecosystems. For example, the system and method, according to the present invention, may be adapted for monitoring small area root and stem gas respiration, respiration from liquid surfaces such as river, lake and ocean surfaces and for monitoring gas flux under snow.

I claim the following:

1. A gas flux measurement system comprising gas collection means, operable to collect gas for analysis, and gas analysing means, operable to analyse the collected gas, wherein the gas collection means comprises at least one elongate tube, the, or each, tube having one or more gas collection openings to provide gaseous communication from outside of the tube to the inside of the tube.

2-20. (canceled)

21. A method of measuring gas flux comprising the steps of:
providing gas collection means comprising at least one elongate tube having one or more gas collection openings; providing gas analysing means; providing gas pumping means; disposing the elongate tube on a surface from which gas flux is to be measured; collecting gas from the said surface and pumping the collected gas through the at least one tube to the gas analysing means; analysing the collected gas and measuring the gas flux.

22-33. (canceled)
